(19) United States
(12) Patent Application Publication
(10) Pub. No.: US 2017/0367710 A1
(43) Pub. Date: Dec. 28, 2017

(54) DOUBLE SIDED OCCLUSIVE DEVICE

(71) Applicant: Cook Medical Technologies LLC, Bloomington, IN (US)

(72) Inventor: Shuo Yang, West Lafayette, IN (US)

(21) Appl. No.: 15/618,185

(22) Filed: Jun. 9, 2017

Related U.S. Application Data
(60) Provisional application No. 62/354,138, filed on Jun. 24, 2016.

(51) Int. Cl. A61B 17/12; A61B 17/00; A61B 90/00

(52) U.S. Cl. CPC A61B 17/12131; A61B 17/0057; A61B 17/12045; A61B 17/12109; A61B 2017/00871; A61B 2017/00637; A61B 2017/00623; A61B 2017/00606; A61B 2017/00597; A61B 2017/00592; A61B 2017/00588; A61B 2017/00526; A61B 2017/00911; A61B 2090/3966; A61B 2017/12054; A61B 2017/00995

(57) ABSTRACT

This application discloses an occlusive device that includes a frame movable between a deliver condition and a blocking condition, the frame including a center region, a plurality of distal arms, a plurality of proximal arms, where both the distal and proximal arms curve away from the center region, with flexible sheet material couple to both the distal and proximal arms that block a lumen when the frame is in the blocking condition. Also disclosed is a method of manufacturing the occlusive device and a medical device incorporating the occlusive device.

### CROSS REFERENCE TO RELATED APPLICATION

This application claims the benefit of U.S. Provisional Application No. 62/354,138 filed Jun. 24, 2016, which is hereby incorporated by reference.

### BACKGROUND

This disclosure relates generally to medical technology and in particular aspects to methods and devices for occluding body passageways.

As further background, there are a variety of reasons why those skilled in the art attempt to occlude or otherwise block fluid flow through vessels and other openings and passageways in the body. In some instances, the desire is to treat an aneurysm, AV fistula or other blood vessel malformation. Although the prior art provides technology in this area, there remain needs for improved and/or alternative devices for occluding or otherwise blocking fluid flow through passageways and openings in the body, as well as methods for preparing and utilizing these devices. The disclosed occlusive device is addressed to those needs.

### SUMMARY

This disclosure details, in certain aspects, unique devices and methods for occluding bodily vessels, and in certain embodiments, vascular vessels. One such device includes a frame and a flexible sheet material that is coupled to the frame. The frame is movable between a first condition that is suitable for transluminal vascular delivery to a vascular site or other vessel site for providing an occluding device, and an expanded second condition that is adapted for deployment at the site. The frame has a proximal end and a distal end and includes a plurality of elongate arms. The arms emanate radially from a common centralized region at the proximal end of the frame, and curve distally to the distal frame end. The expanded second condition includes an expanded frame segment in which the arms are outwardly displaced relative to the common centralized region. The expanded frame segments are spaced proximally and distally from the common centralized region. The flexible sheet material is coupled to the frame such that when the frame is in the expanded second condition at the site, the sheet material is positioned in the vascular or other vessel lumen so as to block fluid flow through the lumen. In some arrangements, the sheet material will extend across essentially the entire vessel lumen when the frame is in the expanded second condition at the site.

The device, in some forms, will have one or more of the following features. The frame can be compressed when in the first condition. The frame can be self-expandable. The expandable material upon expansion at the vascular site can be effective to at least partially move the frame between the first condition and the expanded second condition. The device can be adapted so that the sheet material includes portions located between the arms of the expanded frame segment and inner wall surfaces of the vessel when the frame is in the expanded second condition at the vascular site. The arms of the expanded frame segment can be located between the occluding material and inner wall surfaces of the vessel when the frame is in the expanded second condition at the vascular site. The flexible sheet material can be coupled to the frame such that when the frame moves from the first condition to the expanded second condition at the vascular site, the sheet material is drawn across at least part of the vascular or other vessel lumen.

Other objects, embodiments, forms, features, advantages, aspects, and benefits of the claimed invention shall become apparent from the detailed description and drawings included herein.

### BRIEF DESCRIPTION OF THE DRAWINGS

FIG. 1 is a perspective view of an occlusive device in an expanded state.

FIG. 2 is a perspective view of the FIG. 1 occlusive device in a compressed state.

FIG. 3 is a medical product incorporating the FIG. 1 occlusive device.

FIG. 4 is a partial side view of the FIG. 3 medical product and occlusive device deployed in a vessel.

FIG. 5 is a partial side view of the FIG. 4 medical product with the occlusive device partially deployed into the vessel.

FIG. 6 illustrated one step in a method of deploying the FIG. 1 occlusive device.

FIG. 7 is a side view of the FIG. 1 occlusive device deployed in a vessel.

FIG. 8 illustrates an intermediate manufacturing configuration of the FIG. 1 occlusive device.

FIG. 9 illustrates a preliminary body that can be used in the manufacturing of the FIG. 1 occlusive device.

FIG. 10 illustrates a cross sectional view of the FIG. 9 body.

### DETAILED DESCRIPTION OF THE DRAWINGS

While the claimed invention may be embodied in many different forms, for the purpose of promoting an understanding of the principles of the claimed invention, reference will now be made to the embodiments illustrated in the drawings, and specific language will be used to describe the same. It will nevertheless be understood that no limitation of the scope of the claimed invention is thereby intended. Any alterations and further modifications in the described embodiments and any further applications of the disclosed principles as described herein are contemplated as would normally occur to one skilled in the art to which the claimed invention relates.

As disclosed above, in certain aspects, this disclosure details unique devices for occluding vessels such as vascular vessels. In some forms, these devices are able to move from a first condition to an expanded or otherwise less compact second condition in a vessel so as to at least partially block fluid flow in the vessel, in some cases substantially block fluid flow in the vessel, and in some other cases, to completely prevent fluid from passing through the vessel. Some of these devices are configured to be somewhat compressible so that they can be folded and/or rolled or otherwise compressed into a low-profile condition for delivery through the vasculature, for example, in a catheter lumen. When deployed from the catheter, these devices transform in the vessel to provide a unique blocking arrangement there even when considerable forces act to dispel the device from its deployed location. This disclosure further provides methods for manufacturing these devices, as well as medical products that include such devices enclosed within sterile packaging.

Some disclosed devices sheet or sheet-like material(s) and one or more frame members arranged in and/or around the material. In one particular embodiment, a combination of sheet and frame components itself is effective to essentially block fluid flow through the vessel.

Various types of frame and frame-like elements can be utilized in the disclosed occlusive device. These include single- and multiple-part devices. In some forms, a frame member will include a filament or wire body or other similar frame or frame-like support structure. Frame members, in some embodiments, can be designed to move between a first condition and one or more other conditions, for example, in the case of a frame that is compactable to a compacted, first condition, and when in this compacted condition, is then expandable to an expanded, second condition. In forms where a frame has the capacity to expand, these frames can include those that are considered self-expanding and those that require at least some manipulation in order to expand.

Frames of this sort and other similar support elements useful in the disclosed occlusive device can be constructed using one or more pieces of superelastic wire or any of a variety of other suitable materials described herein or otherwise known to those skilled in the art including MRI compatible materials. Frames and other similar expandable and non-expandable support members, when utilized in the disclosed occlusive device, may be made from metallic or non-metallic material, or both. The non-metallic material can suitably be a synthetic polymeric material, including for example bioresorbable and/or non-bioresorbable plastics. Materials commonly used in medical device construction include biologically compatible metals, e.g., stainless steel, titanium, tantalum, gold, platinum, copper and the like, as well as alloys of these metals; synthetic polymeric materials; low shape memory plastic; a shape-memory plastic or alloy, such as nitinol; and the like.

In certain forms, the frame member is configured to lock itself in position in a vessel to prevent migration of the device after insertion. The frame member and sheet material may be configured to transmit force from blocked fluid flow to the opposite side of the frame to dig outer tip portions of the frame into the vessel wall. Radially extending arms may curve away from the common centralized region to convert a portion of any longitudinal force imparted on the frame into a radial force that encourages arm tips to embed in the vessel wall. The curved arms and the overall geometry of the frame member may also be adapted to resist buckling, permitting comparatively smaller arm geometries to be used in the frame that would be possible with straight arms.

In certain forms, a resilient frame member can be provided in a relaxed condition. The frame can then be deformed (e.g., collapsed, compressed, etc.) from this relaxed, first condition to a deformed, second condition and held there. In this deformed, second condition, the resilient frame is then poised to essentially return to its relaxed, first condition. Illustratively, a frame can be compressed into a compressed condition (e.g., by collapsing radially extending arms toward a centerline) for positioning in a delivery device lumen having a relatively smaller diameter than that which the frame could otherwise fit in its relaxed condition. In this compressed condition, the frame then has the ability to self-expand essentially back to its prior, relaxed condition upon being removed from the delivery device lumen. In other embodiments, frame members and other frame-like elements exhibit little or no resiliency.

In some instances, a frame element may be urged to expand by another device component exerting force on the frame element as the component expands. This can be made to occur with both self-expanding and non-self-expanding frame elements. Frames can be provided and delivered in a contracted state, and then expanded upon the application of a force, e.g. an outward radial force, to the frame.

Frame structures which take on a contracted state, but expand in response to a conditional change, e.g., a change in temperature such as may be incurred in a temperature transition from a first temperature below the body temperature of a patient, to the body temperature of the patient, can also be utilized. Frame members having these or other characteristics may be used in embodiments of the disclosed occlusive device.

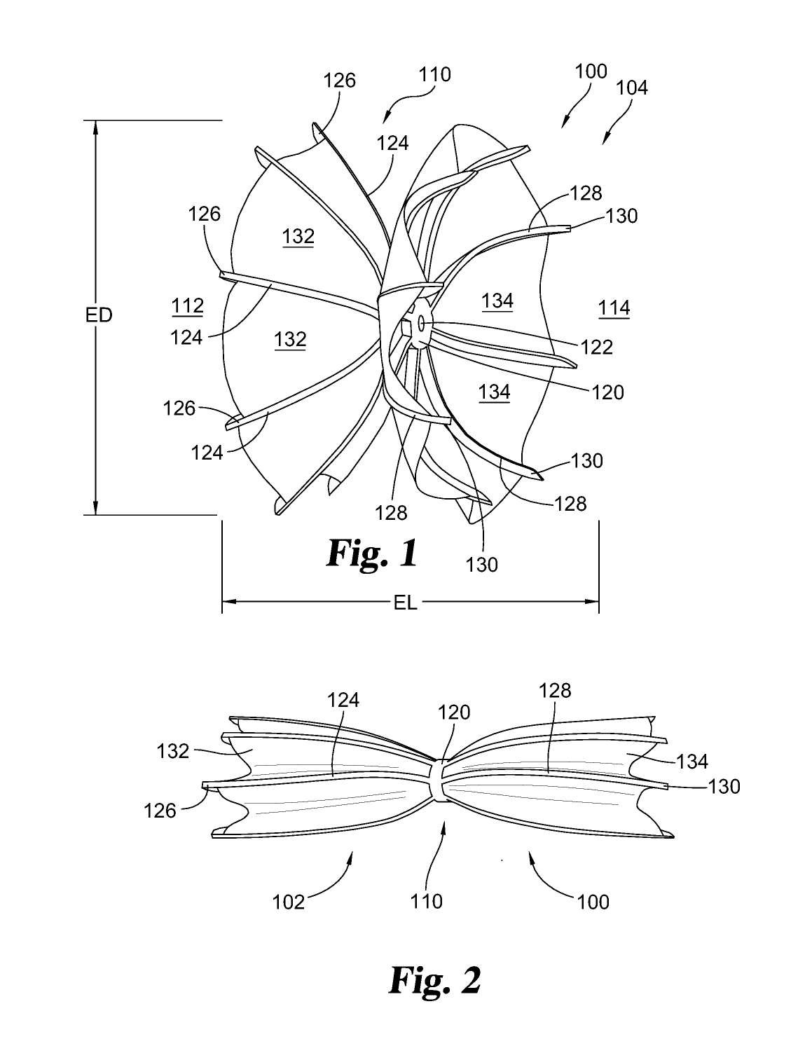

With reference now to FIG. 1, shown is a perspective view of a self-expandable occlusive device 100 useful in certain aspects of the disclosed occlusive device. Occlusive device 100 is movable between compact condition 102 as shown in FIG. 2 and expanded condition 104 as shown in FIG. 1. Occlusive device 100 has a proximal end 112 and a distal end 114 and generally includes frame 110, proximal sheet material 132 and distal sheet material 134.

Frame 110 includes a plurality of proximal arms 124 and distal arms 128. Proximal arms 124 emanate from center region 120, and extend proximally from there to proximal end 112 of the device. Distal arms 128 also emanate from center region 120, and extend distally from there to distal end 114 of the device. Frame 110 is shown in an expanded condition 104 in FIG. 1. As can be seen in FIG. 1, as distal arms 128 extend radially outwardly from center region 120 they curve toward distal end 114 of frame 110. Accordingly, in this disclosed embodiment, the distal end of the frame is open. Similarly, as proximal arms 124 extend radially outwardly from center region 120 they curve toward proximal end 112 of frame 110. Thus the proximal end of frame 110 is also open in this disclosed embodiment.

In the disclosed embodiment, both proximal arms 124 and distal arms 128 initially extend from center region 120 in an almost entirely radial direction, with little extension in either a proximal or distal direction. As both proximal arms 124 and distal arms 128 extend further away from center region 120, both increasingly extend in a proximal or distal direction respectively until, at the end, both proximal arms 124 and distal arms 128 extend relatively more in the proximal or distal direction respective compared to the radial direction. Both proximal arms 124 and distal arms 128 define curved shapes in the disclosed embodiment. Together, proximal arms 124, in the illustrated expanded condition, approximate the shape of a portion of a hollow sphere. Similarly, distal arms 128, in the illustrated expanded condition, also approximate the shape of a portion of a hollow sphere. Together, in the expanded condition, proximal arms 124 and distal arms 128 may approximate the shape of a double-umbrella.

However, either proximal arms 124 or distal arms 128 or both, while exhibiting curvature as they extend away from center region 120 of the frame, may also optionally be made to extend in a variety of other non-straight fashions, or to be straight. When in an expanded condition, frame 110 can be compressed to a compressed condition that is suitable for delivering the frame to a vascular site for providing an occluding device. At the vascular site, frame 110 can be deployed and thus caused or allowed to expand.

Proximal arms 124 include proximal arm ends 126 that may be adapted to embed in the wall of a vessel to secure frame 110 and occlusive device 100 in position. Similarly, distal arms 128 include distal arm ends 130 that may also be adapted to embed in the wall of a vessel to secure frame 110 and occlusive device 100 in position.

In the illustrated embodiment, a sheet or sheet-like material is coupled to the frame such that when the frame moves from a first condition to an expanded second condition at the vascular site, the sheet material will be forced into a blocking arrangement in the lumen with material extending across a substantial portion of the lumen. A sheet material, whether extending fully or partially across the lumen, can occur upstream and/or downstream and/or both of an occluding material in a deployed device.

As shown in FIG. 1, proximal sheet material 132 is coupled to proximal arms 124 and center region 120 to substantially fill the spaces between proximal arms 124 while proximal arm ends 126 may extend beyond proximal sheet material 132. When proximal arm ends 126 embed in a vessel wall, proximal sheet material 132, in combination with frame 110, may substantially occlude the vessel. Proximal sheet material 132 may be integrally unitarily constructed with proximal arms 124 and center region 120 from a single piece of material such as a biodegradable polymer. Alternatively, proximal sheet material 132 may be attached to proximal arms 124 and center region 120 after proximal arms 124 and center region 120 are formed.

Similarly as also shown in FIG. 1, distal sheet material 134 is coupled to distal arms 128 and center region 120 to substantially fill the space between distal arms 128 with distal arm ends 130 extending beyond distal sheet material 134. Distal sheet material 134, in combination with frame 110, may be configured to substantially occlude a vessel when distal arm ends 128 embed in a vessel wall. Distal sheet material 134 may be integrally unitarily construction with distal arms 128 and center region 120 from a single piece of material such as a biodegradable polymer. Alternatively, distal sheet material 134 may be attached to distal arms 128 and center region 120 after distal arms 128 and center region 120 are separately formed.

A first device component can act to shield or otherwise protect a second device component at an occlusion site. Illustratively, an upstream sheet material can reduce or eliminate any deleterious effects of oncoming fluids on a downstream occluding material. Creating these types of protected environments can enhance the overall occlusive result, particularly in situations where the protected component can use that protection to achieve a result that it otherwise would not have been able to achieve. Protection can be provided upstream and/or downstream. In some cases, protection from the forces of oncoming fluids can help prevent migration of the device, or a device component. A protected environment can also increase the amount of time an incorporated substance (e.g., a drug coating, occluding hydrogel, etc.) remains at the occlusion site.

Occlusive device 100, as illustrated in FIG. 1, may be symmetrical about center region 120 such that proximal arms 124 and distal arms 128 are substantially mirror images of each other. Configuring occlusive device 100 symmetrically may advantageously prevent backwards installations and may make occlusive device 100 less expensive to manufacture compared to non-symmetrical devices. However, occlusive device 100 does not have to be symmetrical to provide many of the benefits described herein. Other non-symmetrically configurations of occlusive device 100 are contemplated, for example, varying the rate of curvature of proximal arms 124 compared to distal arms 128. Similarly, the length of proximal arms 124 and distal arms 128 could be different. Proximal arm ends 126 may be configured with blunted ends adapted to not penetrate vessel wall 84 while distal arm ends 130 may be configured to embed in vessel wall 84.

FIG. 2 shows the occlusive device 100 in a compressed condition.

Compressing the device in this manner is useful for fitting the device into a delivery device for transluminal vascular delivery. In such a reduced-diameter condition, the frame and sheet material are both compressed. The flexible sheet material is also compressed or otherwise forced into a more compact condition along with the other components. In a compressed device, the sheet material may roll and/or fold over itself and/or the other device components one or more times. In such a compact condition, the device will be able to travel through bodily passageway which it otherwise would not have been able to pass (or pass as easily).

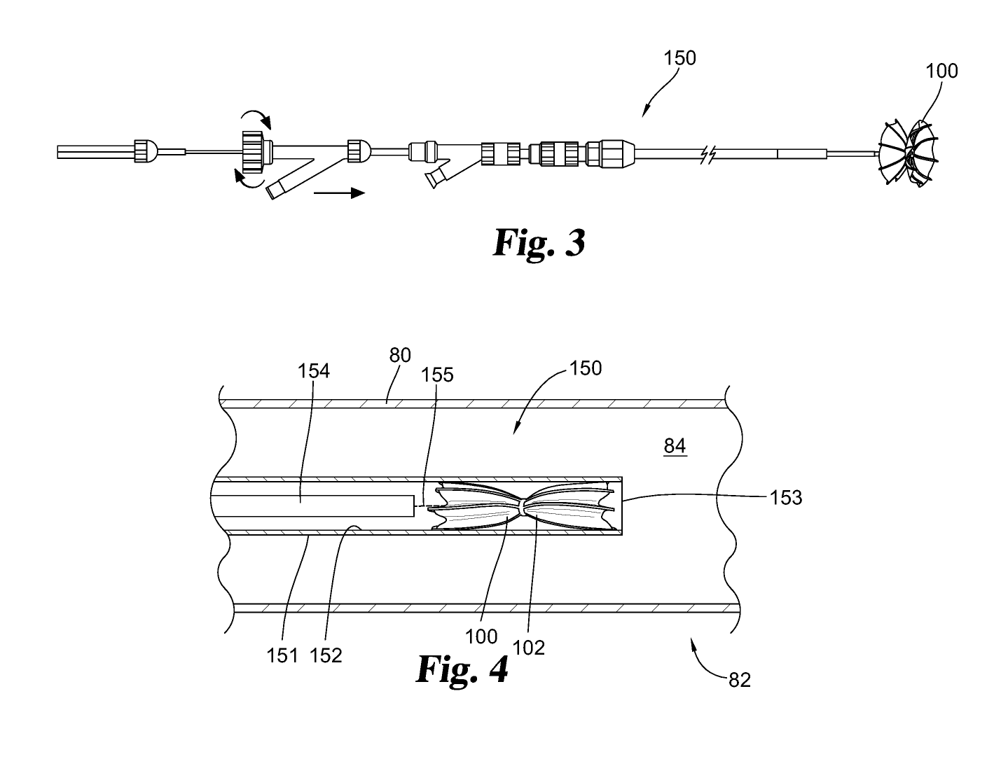

Various types of sheaths and other delivery devices can be utilized with the occlusive devices disclosed herein. FIG. 3 shows a side view of one specific illustrative delivery device that can be used to deliver an occlusion device to a vessel site. In general, these devices will provide space (e.g., a lumen) into which one or more occlusion devices can be placed for delivery into the body. Certain devices may include a lumen communication with a distal, open end. Commercially available catheters and other endoluminally advancable devices may be used in this regard.

FIG. 4 shows a medical product 150 according to another embodiment after it has been advanced to a location in a vessel 80. Medical product 150 includes a delivery sheath 151 having a lumen 152 communicating with a distal end opening 153. Medical product also includes an occlusion device 100 which can exhibit compact configuration 102 for removably positioning the device in the delivery sheath lumen as shown.

Medical product 150 further includes a deployment member 154 that is translatable through lumen 152. In some aspects, a deployment member is a simple pusher that can be used to push an occlusion device from a delivery sheath lumen. Additionally or alternatively, a deployment member may be equipped to somehow engage the occlusion device for moving the occlusion device with respect to the delivery sheath, and potentially also manipulating the occlusion device in the vessel lumen during and/or after deployment. Illustratively, a pusher may provide a mechanism by which to grasp or otherwise grip or capture part of an occlusion device.

Though optional, medical product 150 may include a coupling element 155 that extends between the occlusion device and the deployment member. A coupling element of this sort, when incorporated, can include any suitable adaptation to enable the pusher and occlusion device to be temporarily connected or otherwise united with one another. These include but are not limited to those involving single- and multiple-part coupling mechanisms, grasping devices including lockable and non-lockable forceps, magnetic devices, energizable components, clasps, various bonding materials effective to bond two objects together, and combinations and variations thereof. In some embodiments, a delivery device may include means for visualizing and identifying different device components and their surroundings during deployment.

Upon deployment from the delivery sheath lumen, occlusion device 100 is effective to expand to expanded condition 104 in the vessel as shown in FIGS. 5 and 7. As shown in FIG. 5, upon deployment from delivery sheath 151, distal arms 128 may expand in vessel 80 while proximal arms 124 are still positioned in lumen 152 of delivery sheath 151. Further deployment results in proximal arms 124 also expanding to expanded condition 104 in the vessel as shown in FIG. 7. In this expanded condition, portions of the frame member, whether through self-expansion of the frame member and/or through the force of the expanding occluding material and/or force imparted by movement of a fluid through the vessel, move outward relative to their pre-deployment locations, with the frame member providing a framework in and/or around the expanded sheet material. As portions of the frame move toward the vessel walls, the corresponding attached sheet portions are pulled therealong into a blocking arrangement in the lumen.

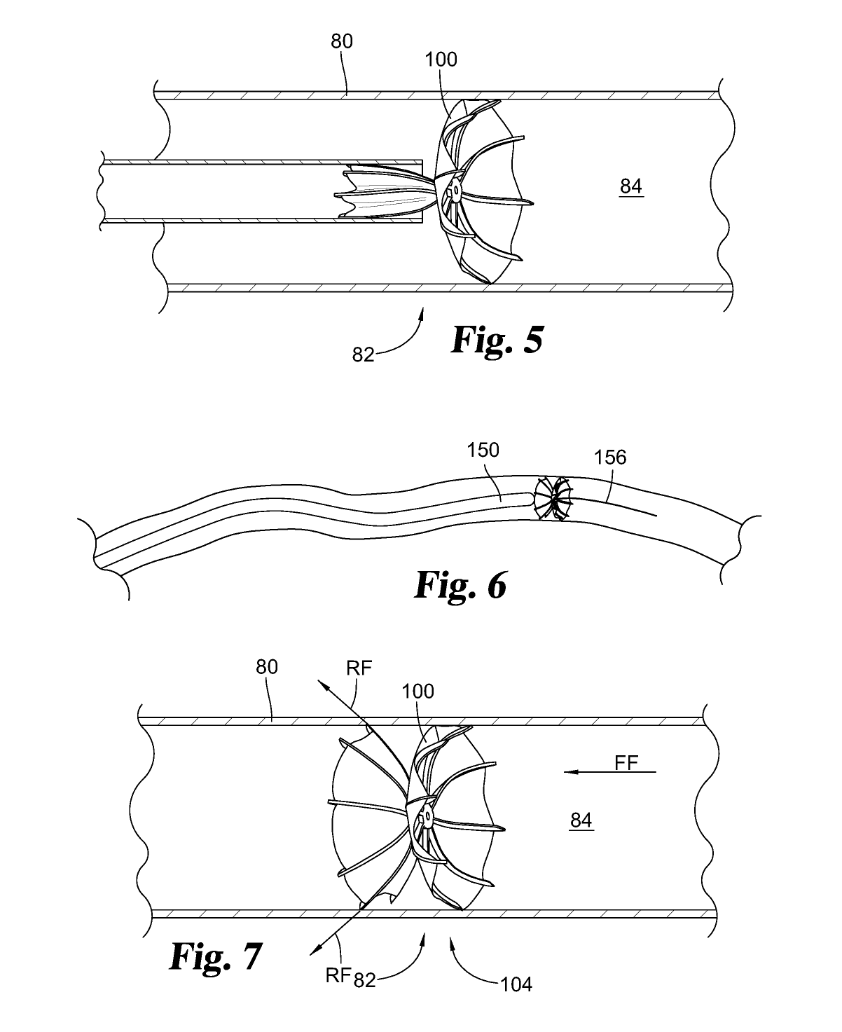

FIG. 6 shows a step in which the occlusion device of FIG. 2 is being deployed in a vessel 80. Prior to this step, the occlusion device is compressed and removably positioned in a lumen of delivery sheath 151. The delivery device is then advanced through the vessel over an emplaced guidewire 156. Once at a desired location, the device is deployed from the delivery device lumen, over the guidewire and into the vessel, whereupon the device expands to block flow through the vessel. While over-the-wire delivery is contemplated, it is not required. The occlusive device may be deployed from delivery sheath 151 either with or without a guidewire passing through hole 122.

Although more than one device can be deployed in a vascular region, in certain peripheral and non-peripheral vascular settings, a single device can be effective to occlude a passage that would have otherwise required many embolic coils, or that cannot be effectively treated with coils or other embolic devices, for example, as occurs with some giant aneurysms and other malformations. Occlusive device 100 can be sized to occlude bodily passageways having diameters ranging from about 2 mm to about 22 mm or more. When a device is used to treat an aneurysm, AV fistula or other blood vessel malformation in the brain or other part of the body, the device may be placed at a variety of locations relative to the malformation. In some instances, a device may be positioned somewhat away from the malformation so that it blocks blood flow to the affected area. Additionally or alternatively, an inventive device can be placed in the affected area, for example, in situations where a device is placed in and around a fistula tract or an aneurysm neck.

Continuing with FIG. 6, the sheet material is coupled to the frame such that when the frame moves from a first condition to an expanded second condition at the vascular site, the sheet material is pulled therealong into a blocking arrangement in the lumen, for example, with the sheet material extending across the lumen between its walls. This blocking arrangement is effective to at least partially block fluid flow through the lumen. In such a blocking arrangement, portions of the sheet material can extend along surfaces of the vessel wall to provide enhanced blocking, and in some cases, completely sealing off of the vessel lumen. The frame member and/or occluding material, upon deployment, will contact the vessel wall. The guidewire and delivery device are then withdrawn, leaving the deployed device behind in the vessel.

Medical product 150 includes a delivery sheath 151, and a deployment member 154 that is translatable through the sheath. Deployment member 154 can be used in a variety of deployment and post-deployment steps. Deployment member 154 may be used to force the device from the sheath, as well as retract the device back into the sheath if desired. The member may also be used to reposition or otherwise manipulate the deployed device in the vessel, for example, by twisting the device and/or moving it back and forth in the vessel. In some cases, portions of the frame or other device adaptations will be effective to abrade inner surfaces of the vessel wall upon contact, and manipulations of this sort can be used to cause a desirable abrasion. Because the point of engagement between the occluding device and the deployment member occurs in a centralized region of the device (e.g., at hole 122), engaging and disengaging the components can occur away from the walls of the vessel.

Deployed devices, in certain embodiments, will provide one or more frame elements at or near the periphery of the device for contacting interior wall surfaces of the vessel for anchoring and other purposes. The distal tips of arms 124 and 128 may include ends 126 and 130 adapted to embed themselves in the vessel wall upon deployment and/or any subsequent repositioning of the device in the vessel. As well, any number of other anchoring adaptations, barbs, ribs, protuberances, and/or other suitable surface modifications can be incorporated into occlusive device 100 to roughen, condition, or otherwise de-epithelialize at least a portion of the vessel wall during and/or after deployment of the device within the vessel. The conditioning of the vessel wall tissue can serve to initiate a localized healing response in patient tissue that can be advantageous in enhancing the ingrowth of patient tissue into occlusive device 100.

Occlusive device 100 may be configured such that the flow of fluid in vessel 80, such as blood, and/or fluid pressure in vessel 80 further secures occlusive device 100 at vascular site 82. Fluid flow direction FF is illustrated in FIG. 7. The fluid flow and/or fluid pressure in vessel 80 exerts a force on occlusive devices 100 that translates through occlusive device 100 resulting in reaction force RF directed along the axis of proximal arm ends 126. Reaction force RF may help embed proximal arm ends 126 into vessel wall 84 thereby locking occlusive device 100 in position and preventing migration of occlusive device 100.

The curved shape of both proximal arms 124 and distal arms 128 may be adapted to permit use of materials that are more flexible/less stiff than other materials. For example, biodegradable polymers can be less stiff than metallic materials such as super elastic wire. The curved shape may permit arms 124 and 128 to be configured having less stiffness that may otherwise be necessary. Distal arms 128 are restrained from radial expansion by vessel wall 84. Fluid flow direction FF exerts force on distal arms 128 that wedges arms 128 between vessel wall 84 and center region 120 and applies a tensile force to distal arms 128 which may prevent buckling of distal arms 128.

Similarly, proximal arms 124 are wedged between vessel wall 84 and center region 120, but the resultant force on proximal arms 124 is compression. The curved shape of proximal arms 124 may be more resistant to buckling compared to proximal arms 124 that are not curved.

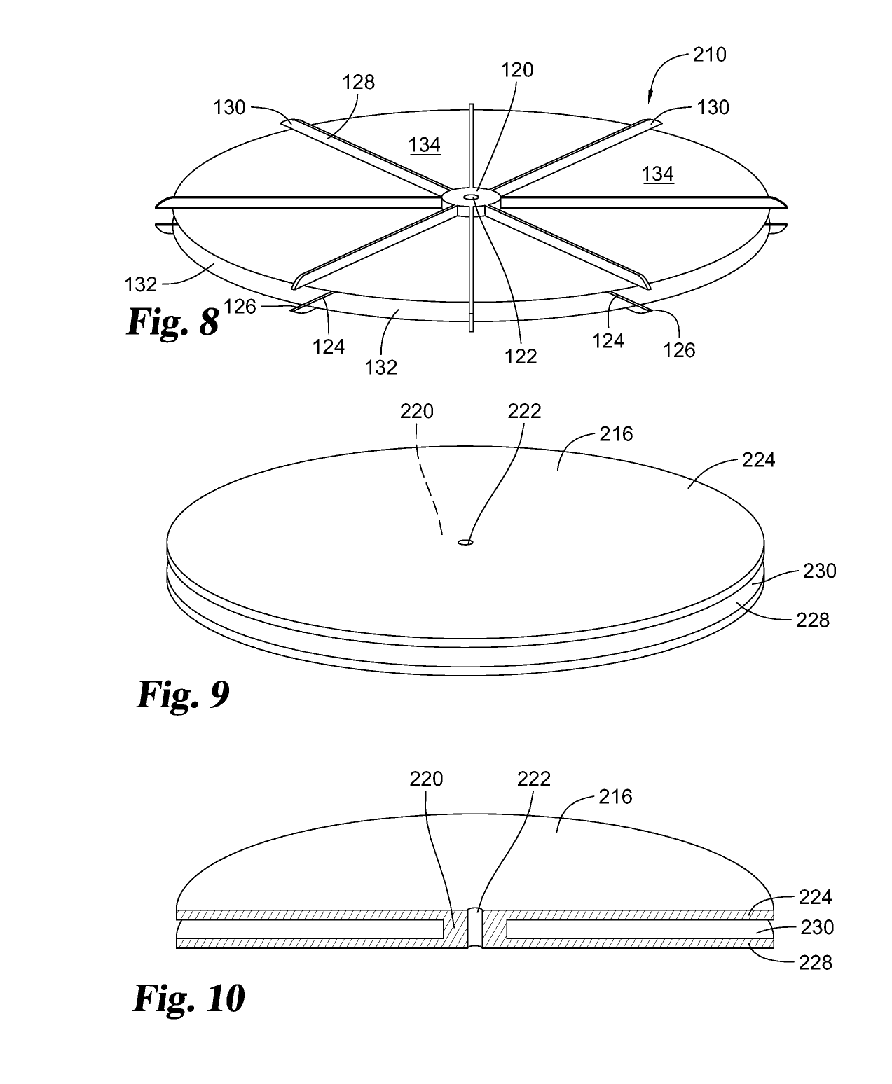

Referring to FIG. 8, preformed body 210 is illustrated. Preformed body 210 illustrates one possible intermediate configuration of occlusive device 100 that could be used in the manufacture of occlusive device 100. Preformed body 210 may include many components of occlusive device 100, although in a different shape with the arms extending radially from a center portion. As shown, preformed body 210 includes center region 120, proximal arms 124, distal arms 128 proximal sheet material 132 and distal sheet material 134. Center region 120 defines hole 122 that passes through center region 120.

Referring to FIGS. 9 and 10, intermediate body 216 is illustrated. Intermediate body 216 may be used in the manufacture of preformed body 210. Intermediate body 216 includes central hub 220, first disk 224 and second disk 228 separated from first disk 224 by gap 230. Central hub 220 also includes hole 222 that passes through central hub 220. Intermediate body 216 may be formed from a single piece of biodegradable polymer.

Preformed body 210 can be manufactured using several methods. Intermediate body 216 can be molded, for example, injection molded, or cut from a piece of biodegradable polymer. Preformed body 210 can be formed from intermediate body 216 by stamping intermediate body 216 with an appropriate die. Stamping may optionally include pre-heating intermediate body 216 to a pliable forming temperature prior to stamping intermediate body 216. After stamping, a cutting process may optionally be used to create a final, accurate geometry as shown in FIG. 8.

Alternatively, preformed body 210 may be molded in a one step process provided that the injection molding process can create reasonably thin sheet materials 132 and 134.

Occlusive device 100 is manufactured from preformed body 210 by deforming distal arms 128 to curve in the distal direction away from center region 120 and deforming proximal arms 124 to curve in the proximal direction away from center region 120. Prior to deforming arms 124 and 128, preformed body 210 may optionally be heated to a pliable forming temperature.

Continuing now with additional discussion of frame shapes and configurations, in some forms, a frame member provides an elongate frame body having either a constant or varying cross-sectional area along its length, or portions thereof. Illustratively, all or part of a frame body can exhibit a generally cylindrical shape, a conical shape, and other suitable shapes including some having tapered and/or non-tapered longitudinal portions. As well, a cross section of a particular frame body portion can exhibit a variety of shapes including some that have rectilinear and/or curvilinear features. Thus, a frame body can include a portion having a generally circular or non-circular (e.g., elliptical, square, star-shaped, hexagonal, etc.) cross section.

The exterior and/or other regions of an occlusion device might be coated with one or more materials or substances such as a drug coating, or the like.

In this regard, in some embodiments, one or more agents or other substances (as described herein) can be conjunctively or cooperatively emplaced within a patient with one or more occlusive implants as are discussed herein. Cooperative emplacement can include the contact of patient tissue with agents before, after, and/or while the occlusive device is implanted in the patient. Such tissue contact of agents can occur in those areas that will become or are in contact with one or more occlusive devices and/or are adjacent to or near the implant or prospective implant location. For example, the agents can be delivered into the patient through a cannulated lumen, such as before an occlusive device is implanted, or can be injected into a patient through a needle and syringe, such as after an occlusive device is implanted. In additional embodiments, the agents can be contained within or on the occlusive device, such as by being applied to an occlusive construct by a physician before implantation occurs, and/or by being doped, bonded, or otherwise contained within a dry occlusive construct, such as can be achieved by soaking a construct in one or more agents and thereafter drying and packaging the construct.

In certain embodiments, a supplemental material will include a substance that is capable of bringing about or inducing constriction, spasm, or closure in a bodily vessel of a patient and/or causing the de-epithelialization or inflammation (either dilative or constrictive), and/or otherwise initiating a healing response in patient tissue, such as a wall segment of a venous vessel. Such agents can include any suitable vasoconstrictive agent, sclerosive agent, thrombogenic agent, inflammatory agent, hypercoagulable agent, or any suitable combination of one or more of any of the above or other suitable agents. For example, suitable vasoconstrictive agents can include any suitable alpha adrenergic direct or indirect agonist, such as norepinephrine, epinephrine, phenylephrine, and/or cocaine, or lidocaine, hypertonic saline, or any suitable combination thereof. Illustrative sclerosive agents can include, for example, polidocanol, sodium tetradecyl sulfate, e.g. SOTRADECOL®, morrhuate sodium, ethanolamine oleate, tetradecyl sulfate, tetracycline, glycerin, hypertonic glucose, talc, acetic acid, alcohol, bleomycin, picibanil, ethibloc, deoxycycline, and/or any suitable microfoam that contains a sclerosive agent, such as VARISOLVE®, manufactured by Provensis, Ltd. of London, England, or any other suitable agent as disclosed in U.S. Pat. Nos. 5,676,962 and/or 6,572,873, for example. In some aspects, an anesthetic agent may be added to a sclerosant agent mixture or other fill material.

Turning now to a more detailed discussion of materials that can be used in the manufacture of certain inventive devices, occluding devices of the claimed invention can incorporate naturally derived and/or non-naturally derived materials. Illustratively, sheet form and non-sheet form materials, such as sheet 33 may comprise one or more of a variety of synthetic polymeric materials including but not limited to bioresorbable and/or non-bioresorbable polymers. Bioresorbable, or bioabsorbable polymers that may be used include, but are not limited to, poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(glycolic acid-co-trimethylene carbonate), polyhydroxyalkanaates, polyphosphoester, polyphosphoester urethane, poly(amino acids), cyanoacrylates, poly(trimethylene carbonate), poly(iminocarbonate), copoly(ether-esters) (e.g., PEO/PLA), polyalkylene oxalates, and polyphosphazenes. These or other bioresorbable materials may be used, for example, where only a temporary blocking or closure function is desired, and/or in combination with non-bioresorbable materials where only a temporary participation by the bioresorable material is desired.

Non-bioresorbable, or biostable polymers that may be used include, but are not limited to, polytetrafluoroethylene (PTFE) (including expanded PTFE), polyethylene terephthalate (PET), polyurethanes, silicones, and polyesters and other polymers such as, but not limited to, polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins, polycarbonates; polyoxymethylenes; polyimides; polyethers; epoxy resins, polyurethanes; rayon; and rayon-triacetate.

As well, inventive devices can incorporate biocompatible materials derived from a number of biological polymers, which can be naturally occurring or the product of in vitro fermentation, recombinant genetic engineering, and the like. Purified biological polymers can be appropriately formed into a substrate by techniques such as weaving, knitting, casting, molding, and extrusion. Suitable biological polymers include, without limitation, collagen, elastin, keratin, gelatin, polyamino acids, polysaccharides (e.g., cellulose and starch) and copolymers thereof.

In certain embodiments, one or more device components will be comprised of a remodelable material. Particular advantage can be provided by devices that incorporate a remodelable collagenous material. Such remodelable collagenous materials, whether reconstituted or naturally-derived, can be provided, for example, by collagenous materials isolated from a warm-blooded vertebrate, and especially a mammal. Such isolated collagenous material can be processed so as to have remodelable, angiogenic properties and promote cellular invasion and ingrowth. Remodelable materials may be used in this context to promote cellular growth on, around, and/or within vessels and other bodily spaces into which inventive devices are implanted.

While useful to provide occlusions and blockages in arteries and veins and other openings and passageways in the vasculature, inventive devices can be adapted and used to occlude, block, fill, etc. a variety of suitable passageway and open spaces in the body including those in non-vascular locations. In some instances, an inventive device will be configured for placement in a naturally occurring location in the body, for example, in a native lumen or other open space in a bodily system, e.g., in an organ or other component of the circulatory, respiratory, digestive, urinary and reproductive, sensory, or endocrine systems. In certain aspects, a space to be occluded is one that exists naturally in the body but relates to a disease, defect, deformation, etc. Alternatively, an opening or passage to be filled may be one resulting from an intentional or unintentional trauma to the body including but not limited to some relating to vehicular accidents, gunshots and other similar wounds, etc., as well as some formed by passage of a medical instrument (e.g., a needle, trocar, etc.) through cutaneous, subcutaneous, and/or intracutaneous tissue.

The disclosed device also includes, in certain aspects, medical products that include a radiopaque element such as but not limited to a radiopaque coating, attached radiopaque object, or integrated radiopaque substance. Any suitable radiopaque substance, including but not limited to, tantalum such as tantalum powder, can be incorporated into a disclosed medical product. Other radiopaque materials comprise bismuth, iodine, and barium, as well as other suitable markers.

In additional embodiments, the claimed invention provides medical products that include means or devices as described herein for delivering occlusion devices into and otherwise providing occlusion in the vasculature, and written materials including instructions for use of the means or devices to deliver occlusion devices into and otherwise provide occlusion in the vasculature. The products can include the means or devices packaged together with the instructions, e.g. in sterile medical packaging. Related embodiments of the claimed invention include methods for distributing such means or devices, or otherwise conducting business, which include distributing such means or devices for delivering occlusion devices into and otherwise providing occlusion in the vasculature, and also distributing information relating the use of such means or devices for delivering occlusion devices into and otherwise providing occlusion in the vasculature. Such information can be distributed packaged with the means or device, or separately, e.g. including information or instructions available on a communication network, including a global computer communication network such as the internet.

The disclosed medical product can be included, in certain aspects, in a line of medical products, wherein a disclosed medical product includes one or more devices, apparatuses or systems in a sealed package. In some forms, medical products are provided that include one or more occlusion devices such as any of those described herein, and potentially also a suitable delivery apparatus or other delivery instrumentation, enclosed within sterile medical packaging. Illustratively, such a medical product can have packaging including a backing layer and a front film layer that are joined by a boundary of pressure-adhesive as is conventional in medical packaging, wherein the contents of the packaging are sealed between the backing layer and front film layer. Sterilization of such a medical product may be achieved, for example, by irradiation, ethylene oxide gas, or any other suitable sterilization technique, and the materials and other properties of the medical packaging will be selected accordingly.

Additionally, the package can include indicia to communicate the contents of the package to a person, machine, computer, and/or electronic device. Such indicia may include the dimensions of, the type of materials used to form, and/or other useful information regarding the contents of the package. In certain embodiments, the contents are packaged for sale with instructions for use. For example, in certain embodiments, a medical product includes at least one occlusion device and delivery instrumentation sealed within a sterile package, wherein the packaging can have visible indicia identifying the contents as suitable for providing occlusion in the vasculature, and/or can contain or otherwise be associated with printed materials identifying the contents as such and including information concerning their use.

Any theory, mechanism of operation, proof, or finding stated herein is meant to further enhance understanding of the disclosed occlusion device, and is not intended to limit the claimed invention in any way to such theory, mechanism of operation, proof, or finding. While the claimed invention has been illustrated and described in detail in the drawings and foregoing description, the same is to be considered as illustrative and not restrictive in character, it being understood that only selected embodiments have been shown and described and that all equivalents, changes, and modifications that come within the spirit of the disclosed occlusive device as defined herein or by the following claims are desired to be protected.

What is claimed is:

1. An occlusive device for occluding a vessel, the device comprising:
a frame movable between a first condition suitable for transluminal delivery to a vessel site for providing an occluding device, and an expanded second condition adapted for deployment at the vessel site, the frame having a proximal end and a distal end and including:
a common centralized region;
a plurality of first elongated arms that emanate from the common centralized region and extend distally to the distal frame end;
a plurality of second elongated arms that emanate from the common centralized region and extend proximally to the proximal frame end, wherein, in the expanded second condition, the first arms are outwardly displaced relative to the common centralized region and curve distally from the common centralized region, wherein, in the expanded second condition, the second arms are outwardly displaced relative to the common centralized region and curve proximally from the common centralized region;
a first flexible sheet material coupled to the first arms such that when the frame is in the expanded second condition at the vessel site, the first sheet material is positioned in the vessel lumen so as to block fluid flow through the lumen; and
a second flexible sheet material coupled to the second arms such that when the frame is in the expanded second condition at the vessel site, the second sheet material is positioned in the vessel lumen so as to block fluid flow through the lumen.

2. The occlusive device of claim 1, wherein at least some of the plurality of first elongated arms comprise arm ends adapted to embed in a wall of the vessel.

3. The occlusive device of claim 1, wherein the common centralized region, the plurality of first elongated arms and the plurality of second elongated arms are unitarily constructed from a single piece of material.

4. The occlusive device of claim 3, the single piece of material is a biodegradable polymer.

5. The occlusive device of claim 1, wherein the common centralized region, the plurality of first elongated arms, the plurality of second elongated arms, the first flexible sheet material and the second flexible sheet material are unitarily constructed from a single piece of material.

6. The occlusive device of claim 5, the single piece of material is a biodegradable polymer.

7. The occlusive device of claim 1, wherein the first and second flexible sheet material is coupled to the arms so as to each be drawn across the vessel lumen when the frame moves from the first condition to the expanded second condition at the vessel site.

8. The occlusive device of claim 1, wherein, in the second expanded condition, the first arms, the common centralized region and the first sheet define a shape that approximates a portion of a hollow sphere.

9. The occlusive device of claim 8, wherein, in the second expanded condition, the second arms, the common centralized region and the second sheet define a shape that approximates a portion of a hollow sphere.

10. The occlusive device of claim 1, wherein the common centralized region defines a hole that passes through the common centralized region.

11. The occlusive device of claim 1, wherein, in the second expanded condition, a maximum diameter of the frame is greater than a total longitudinal length of the frame.

12. The occlusive device of claim 1, wherein the plurality of first elongated arms and the plurality of second elongated arms are symmetric about the common centralized region.

13. A method for manufacturing an occlusive device for occluding a vessel, the method comprising:
forming a preformed body that comprises a common centralized region, a plurality of first elongated arms that radiate from a first end of the common centralized region, a plurality of second elongated arms that radiate from a second end of the common centralized region opposite the first end;
deforming the plurality of first elongated arms to curve away from the common centralized region in a first direction; and
deforming the plurality of second elongated arms to curve away from the common centralized region in a second direction opposite the first direction.

14. The method of claim 13, wherein the preformed body further comprises a first flexible diaphragm that extends between each of the plurality of first elongated arms and a second flexible diaphragm that extends between each of the plurality of second elongated arms.

15. The method of claim 13, further comprising injection molding the preformed body from a biodegradable polymer.

16. The method of claim 13, further comprising stamping a piece of biodegradable polymer to form the preformed body.

17. The method of claim 16, wherein the piece of biodegradable polymer includes a central hub and two spaced apart and parallel disks of material that radiate from the central hub.

18. The method of claim 13, wherein the common centralized region defines a hole that passes through the common centralized region.

19. The method of claim 13, further comprising cutting the preformed body to create a final geometry before deforming the first or second elongated arms.

20. The method of claim 13, further comprising heating the preformed body to a pliable forming temperature before deforming the first or second elongated arms.

21. A medical product for delivering an occlusive device to a vessel, the medical product comprising:
an endoluminally advancable delivery device having a lumen; and
the occlusive device of claim 1 removably positioned in the lumen of the delivery device, the occlusive device being adapted for deployment from the delivery device lumen in the vessel for occluding the vessel.
